(19) United States
(12) Patent Application Publication
(10) Pub. No.: US 2021/0030847 A1
(43) Pub. Date: Feb. 4, 2021

(54) TREATMENT OF POST-BARIATRIC HYPOGLYCEMIA USING MINI-DOSE STABLE GLUCAGON

(71) Applicants: XERIS PHARMACEUTICALS, INC., Chicago, IL (US); Joslin Diabetes Center, Inc., Boston, MA (US)

(72) Inventors: Brett NEWSWANGER, Chicago, IL (US); Steven J. PRESTRELSKI, Chicago, IL (US); Mary-Elizabeth PATTI, Boston, MA (US)

(73) Assignees: XERIS PHARMACEUTICALS, INC., Chicago, IL (US); Joslin Diabetes Center, Inc., Boston, MA (US)

(21) Appl. No.: 16/964,124

(22) PCT Filed: Jan. 23, 2019
(86) PCT No.: PCT/US2019/014815

§ 371 Date: Jul. 22, 2020

Related U.S. Application Data
(60) Provisional application No. 62/620,861, filed on Jan. 23, 2018.

(51) Int. Cl. A61K 38/26; A61P 3/08

(52) U.S. Cl. CPC A61K 38/26; A61K 9/0021; A61P 3/08

(57) ABSTRACT

Post-bariatric hypoglycemia (PBH) is an increasingly-recognized complication of gastric bypass surgery. Current therapeutic options have suboptimal efficacy. Small doses of stable liquid glucagon can be used to treat or prevent post-bariatric hypoglycemia.

### STATEMENT REGARDING FEDERALLY FUNDED RESEARCH

This invention was made with government support under Grant No. R44DK107114 awarded by U.S. Department of Health and Human Services. The government has certain rights in the invention.

### FIELD OF THE INVENTION

The present invention is directed to the field of weight loss medicine and surgery. In particular aspects the invention is directed to methods for treating post-bariatric hypoglycemia (PBH).

### BACKGROUND OF THE INVENTION

Abnormal increases in insulin secretion can lead to profound hypoglycemia or low blood sugar, a state that may result in significant morbidities including seizures and cerebral damage. Drug-induced hypoglycemia can result from administration of sulfonylurea drugs or from an overdose of insulin. A number of medical conditions feature non-drug-induced, endogenous hyperinsulinemic hypoglycemia, such as hyperinsulinemic hypoglycemia following gastric bypass surgery.

Hypoglycemia results in a variety of symptoms including; lack of coordination, confusion, loss of consciousness, seizures, and even death. Most episodes of mild hypoglycemia are effectively self-treated by ingestion of glucose tablets or other carbohydrate containing drinks or snacks. More severe symptomatic hypoglycemia also can be treated with oral carbohydrate ingestion. However, when the hypoglycemic patient cannot take oral glucose supplements, because of confusion, unconsciousness or other reasons, parenteral therapy is required. As a non-hospital rescue procedure, injection of the hyperglycemic hormone, glucagon, is sometimes employed, either subcutaneously or intramuscularly by the patient himself or an associate of the patient who has been trained to recognize and treat severe hypoglycemia.

Post-prandial hypoglycemia (PPH) has recently been observed as a side effect or complication of gastric bypass surgery (post-bariatric patients) (Singh et al., Diabetes Spectrum 25:217-21, 2012; Patti et al., Diabetotogia 48:2236-40, 2005; Service et al. N Engl J Med 353:249-54, 2005), including after the common procedure of Roux-en-Y gastric bypass (RYGB). A commonly observed side effect of gastric bypass surgery is “dumping,” which is a consequence of the ingestion of simple sugars and rapid emptying of food into the small intestine. This is often characterized by vasomotor symptoms (e.g., flushing, tachycardia), abdominal pain, and diarrhea (Singh et al., Diabetes Spectrum 25:217-21, 2012; Mathews et al., Surgery 48:185-94, 1960). Late dumping can occur up to a few hours after eating and results from the insulin response to hyperglycemia resulting from rapid absorption of simple sugars from the proximal small intestine. In contrast to dumping, which is noted soon after surgery and improves with time, hyperinsulinemic hypoglycemia presents several months to years (usually around 1 year, up to 3 years) after gastric bypass surgery. This syndrome is differentiated from dumping by onset of severe postprandial neuroglycopenia, which is typically absent in dumping, as well as pancreatic nesidioblastosis (islet cell enlargement, β-cells budding from ductal epithelium, and islets in apposition to ducts). Unlike with dumping, nutrition modification does not alleviate the symptoms of post-prandial hypoglycemia (PPH).

There remains a need for additional methods for treating post-prandial hypoglycemia in post-bariatric surgery patients.

### SUMMARY

Post-bariatric hypoglycemia (PBH) is an increasingly-recognized complication of gastric bypass surgery. Current therapeutic options have suboptimal efficacy. Certain embodiments of the invention are directed to the administration of lower, more physiologic doses of glucagon or a glucagon analog to ameliorate PBH. The methods and compositions described herein provide a more effective strategy to reduce the likelihood and severity of hypoglycemia in patients with or at risk of developing PBH while also preventing rebound hyperglycemia.

Certain embodiments are directed to methods for treating, ameliorating, or preventing PBH by administering to a subject in need thereof a formulation(s) of glucagon or glucagon analog (e.g., dasiglucagon) in an amount effective to treat, ameliorate, or prevent PBH. In certain aspects the subject is determined to be at risk of developing post-prandial bariatric hypoglycemia (PBHS). The subject can be administered a glucagon or a glucagon analog composition 1, 5, 10, 15, 20, 25, 30, 35, 40, 45, 50, 55, 60, 65, 70, 75, 80, 85, 90 minutes, including all values and ranges there between, prior to, during, and/or after a meal; or when blood glucose levels indicate the need for a dose. In certain aspects the subject is a diabetic subject. In a further aspect, 25, 50, 75, 100, 125, 150, 175, 200, 225, 250, 275, to 300 μg of glucagon or glucagon analog are administered, in certain aspects 150±50 μg of glucagon or a glucagon analog are administered. The glucagon or glucagon analog can be administered as a bolus or as an infusion over time, e.g., infusion time of 90 seconds to 30 minutes. In certain aspects the glucagon or glucagon analog are administered using a glucagon pump or injection device. In certain aspects a second dose of glucagon or glucagon analog can be administered after a first dose, a meal, and/or when blood glucose levels indicate the need for a second dose. In certain aspects blood glucose is being continuously or frequently monitored. The second dose can be a dose of 25, 50, 75, 100, 125, 150, 175, 200, 225, 250, 275, to 300 μg of glucagon or glucagon analog, in certain aspects 150±50 μg. The second dose can be administered 10, 15, 20, 25, 30, 40, 50, 60, 70, 80, 90, 100, 110, 120, 130, 140, 150, 160, 170, 180, 190, 200, 250 minutes or more after a first dose, a meal, or when blood glucose levels indicate a need. In other aspects, independently of or in conjunction with blood glucose levels the second dose can be administered when a certain blood glucose level is measured or a glucose level threshold is being approached or reached. In certain aspect a second dose is administered after a blood glucose level of 90, 80, 70, 60, 50 mg/dL or lower has been measured. A first, second or subsequent dose can be administered when the blood glucose levels fall to below 100, 90, 80, 70, 60, or 50 mg/dL within a certain period of time (e.g., within 90, 80, 70, 60, 50, 40, 30, 25, 20, 15, 10, 9, 8, 7, 6, 5, 4, 3, 2, or 1 minute(s)) after a meal. In certain aspects the targeted blood glucose levels, i.e., those that indicate a risk of hypoglycemia (e.g., a falling blood glucose level of 90, 80, 70, 60, or 50 mg/dL), are maintained or decreases over 0.5, 1, 10, or 20 minutes. In certain aspects 1, 2, 3, 4, or more doses can be administered after a meal. In particular aspects 300 μg of glucagon is administered approximately 90 minutes after the meal. In certain instance the dose will be determined by the absolute blood glucose value in combination with the rate of decrease of blood glucose value, both of which can be provided by a continuous glucose monitor. In certain instance glucagon can be administered approximately or about 15 min before blood glucose reaches 70 mg/dl. If a high rate of decrease is determined, anticipated, or has occurred before then the threshold glucose levels can be about around 100 mg/dl.

A suitable dosage of glucagon or glucagon analog may be administered in the methods of the present invention. The dosage administered will, of course, vary depending upon known factors, such as the pharmacodynamic characteristics of the particular compound, salt, or combination; the age, health, or weight of the subject; the nature and extent of symptoms; the metabolic characteristics of the drug and patient, the kind of concurrent treatment; the frequency of treatment; or the effect desired. In certain aspects hypoglycemia can be treated by administering an effective amount of glucagon.

Certain embodiments are directed to administering a glucagon, glucagon analog, or salt thereof to a subject at risk of post bariatric hypoglycemia. A subject at risk of post bariatric hypoglycemia can be a patient having a decreasing post-prandial blood glucose level below 90, 80, 70, 60, or 50 mg/dL. Formulations can include glucagon, glucagon analog, or a salt thereof at a concentration of at least, at most, or about 0.1, 1, 10, 50, or 100 mg/mL to 150, 200, 300, 400, or 500 mg/ml or up to the solubility limit of the glucagon, glucagon analog, or a salt thereof in the aprotic polar solvent system. In certain aspects, the aprotic polar solvent system can comprise at least one ionization stabilizing excipient that provides physical and chemical stability to the glucagon, glucagon analog, or salt thereof. The formulation can include an ionization stabilizing excipient at a concentration of at least, at most, or about 0.01, 0.1, 0.5, 1, 10, or 50 mM to 10, 50, 75, 100, 500, 1000 mM, or up to the solubility limit of the ionization stabilizing excipient in the aprotic polar solvent system. In certain aspects the ionization stabilizing excipient concentration is between 0.1 mM to 100 mM. In certain embodiments the ionization stabilizing excipient may be a suitable mineral acid, such as sulfuric or hydrochloric acid. In certain aspects the ionization stabilizing excipient may be an organic acid, such as an amino acid, amino acid derivative, or the salt of an amino acid or amino acid derivative (examples include glycine, trimethylglycine (betaine), glycine hydrochloride, and trimethylglycine (betaine) hydrochloride). In a further aspect the amino acid can be glycine or the amino acid derivative trimethylglycine. In further aspects the aprotic solvent system comprises or is DMSO. The aprotic solvent can be deoxygenated, e.g., deoxygenated DMSO.

In certain embodiments the formulation may be prepared by first adding the ionization stabilizing excipient to the aprotic polar solvent system, followed by addition of the glucagon, glucagon analog, or salt thereof. Alternatively, the glucagon, glucagon analog, or salt thereof may initially be solubilized in the aprotic polar solvent system followed by addition of the ionization stabilizing excipient. In a further aspect, the ionization stabilizing excipient and the glucagon, glucagon analog, or salt thereof may be solubilized simultaneously in the aprotic polar solvent system.

### DEFINITIONS

The term “glucagon” refers to the glucagon peptide, analogs thereof, and salt forms of either thereof.

“Analogue” and “analog,” when referring to a peptide or protein, refers to a modified peptide or protein wherein one or more amino acid residues of the peptide or protein have been substituted by other amino acid residues, or wherein one or more amino acid residues have been deleted from the peptide or protein, or wherein one or more amino acid residues have been added to the peptide or protein, or any combination of such modifications. Such addition, deletion, or substitution of amino acid residues can take place at any point, or multiple points, along the primary structure comprising the peptide, including at the N-terminal of the peptide or protein and/or at the C-terminal of the peptide or protein. “Analogue” or “analog” also includes functional analogs or mimetics/peptomimetics.

“Derivative,” in relation to a parent peptide or protein, refers to a chemically modified parent peptide or protein or an analog thereof, wherein at least one substituent is not present in the parent peptide or protein an analog thereof. One such non-limiting example is a parent peptide or protein which has been covalently modified. Typical modifications are amides, carbohydrates, polysaccharides, glycans, alkyl groups, acyl groups, esters, pegylations and the like.

As used herein, the term “post-prandial” refers to the time after a meal. As used herein, the term “post-prandial symptoms” refers to symptoms that occur after a subject has ingested a meal.

A peptide's “optimal stability and solubility” refers to the pH environment wherein solubility of the peptide is high (at or near the maximum on a solubility' versus pH profile, or suitable for the requirements of the product) and its degradation minimized relative to other pH environments. Notably, a peptide may have more than one pH of optimal stability and solubility. A person having ordinary skill in the art can easily ascertain a given peptide's optimal stability and solubility by referencing literature or by performing assays.

The term “dissolution” as used herein refers to a process by which a material(s) in a gas, solid, or liquid state becomes a solute(s), a dissolved component(s), of a solvent, forming a solution of the gas, liquid, or solid in the solvent. In certain aspects a therapeutic agent (e.g., glucagon or a glucagon analog) or an excipient, e.g., an ionization stabilizing excipient, is present in an amount up to its solubility limited or is fully solubilized. The term “dissolve” refers to a gas, liquid, or solid becoming incorporated into a solvent to form a solution.

The term “excipient” as used herein refers to a natural or synthetic substance formulated alongside the active or therapeutic ingredient (an ingredient that is not the active ingredient) of a medication, included for the purpose of stabilization, bulking, or to confer a therapeutic enhancement on the active ingredient in the final dosage form, such as facilitating drug absorption, reducing viscosity, enhancing solubility, adjusting tonicity, mitigating injection site discomfort, depressing the freezing point, or enhancing stability. Excipients can also be useful in the manufacturing process, to aid in the handling of the active substance concerned such as by facilitating powder flowability or non-stick properties, in addition to aiding in vitro stability such as prevention of denaturation or aggregation over the expected shelf life.

The term “therapeutic agent” encompasses proteins, peptides, and pharmaceutically acceptable salts thereof. Useful salts are known to those skilled in the art and include salts with inorganic acids, organic acids, inorganic bases, or organic bases. Therapeutic agents useful in the present invention are those protein and/or peptide that affect a desired, beneficial, and often pharmacological, effect upon administration to a human or an animal, whether alone or in combination with other pharmaceutical excipients or inert ingredients.

The term “peptide” and “peptide compound” refers to amino acid or amino acid-like (peptidomimetics) polymers of up to about 200 amino acid residues bound together by amide (CONH) or other linkages. In certain aspects a peptide can be up to 150, 100, 80, 60, 40, 20, or 10 amino acids. “Protein” and “protein compound” refer to polymers of greater than 200 amino acid residues bound together by amide linkages. Analogs, derivatives, agonists, antagonists, and pharmaceutically acceptable salts of any of the peptide or protein compounds disclosed here are included in these terms. The terms also include peptides, proteins, peptide compounds, and protein compounds that have D-amino acids, modified, derivatized, or naturally occurring amino acids in the D- or L-configuration and/or peptomimetic units as part of their structure.

“Single-phase solution” refers to a solution prepared from a therapeutic agent that is dissolved in a solvent, or solvent system (e.g., mixture of two or more solvents), wherein the therapeutic agent is completely dissolved in the solvent and there is no longer particulate matter visible, such that the solution can be described as optically clear. A single-phase solution may also be referred to as a “single-phase system,” and is distinguished from a “two-phase system” in that the latter is comprised of particulate matter (e.g., powder) suspended in a fluid.

“Inhibiting” or “reducing” or “ameliorating” or any variation of these terms includes any measurable decrease or complete inhibition to achieve a desired result.

“Ameliorating” or any variation of these terms includes any improvement of benefit to a subject in regard to a targeted condition.

“Effective” or “treating” or “preventing” or any variation of these terms means adequate to accomplish a desired, expected, or intended result.

“Chemical stability,” when referring to a therapeutic agent, refers to an acceptable percentage of degradation products produced by chemical pathways such as oxidation and/or hydrolysis and/or fragmentation and/or other chemical degradation pathways. In particular, a formulation is considered chemically stable if no more than about 20% breakdown products are formed after one year of storage at the intended storage temperature of the product (e.g., room temperature); or storage of the product at 25° C. at 60% relative humidity for one year; or storage of the product at 40° C. at 75% relative humidity for one month, and preferably three months in some embodiments, a chemically stable formulation has less than 20%, less than 15%, less than 10%, less than 5%, less than 4%, less than 3%, less than 2%, or less than 1% breakdown products formed after an extended period of storage at the intended storage temperature of the product.

“Physical stability,” when referring to a therapeutic agent, refers to an acceptable percentage of aggregates (e.g., dimers, trimers and larger forms) being formed. In particular, a formulation is considered physically stable if no more that about 15% aggregates are formed after one year of storage at the intended storage temperature of the product (e.g., room temperature); or storage of the product at 25° C. at 60% relative humidity for one year; or storage of the product at 40° C. at 75% relative humidity for one month, and preferably three months. In some embodiments, a physically stable formulation has less than less than 15%, less than 10%, less than 5%, less than 4%, less than 3%, less than 2%, or less than 1% aggregates formed after an extended period of storage at the intended storage temperature of the product.

“Stable formulation” refers to a formulation where at least about 65% of the therapeutic agents (e.g., peptides or salts thereof) remain chemically and physically stable after two months of storage at room temperature. Particularly preferred formulations are those in which at least about 80%, 85%, 90%, 91%, 92%, 93%, 94%, 95%, 96%, 97%, 98%, or 99% chemically and physically stable therapeutic agent remains under these storage conditions. Especially preferred stable formulations are those which do not exhibit degradation after sterilizing irradiation (e.g., gamma, beta, or electron beam).

As used herein, “parenteral administration” refers to administration of a therapeutic agent to a patient via a route other than the alimentary canal—any administration that is not by way of the digestive tract.

As used herein, “parenteral injection” refers to the administration of therapeutic agents (e.g., peptides or small molecules) via injection under or through one or more layers of skin or mucus membranes of an animal, such as a human. Standard parenteral injections are given into the subcutaneous, intramuscular, or intradermal region of an animal or subject, as a human. These deep locations are targeted because the tissue expands more easily relative to shallow dermal sites to accommodate injection volumes required to deliver most therapeutic agents, e.g., 0.1 to 3.0 cc (mL).

The term “intracutaneous” encompasses administration into the epidermal, dermal or subcutaneous skin layer.

As used herein, the term “aprotic polar solvent” refers to a polar solvent which does not contain acidic hydrogen and thus does not act as a hydrogen bond donor. Polar aprotic solvents include, but are not limited to dimethylsulfoxide (DMSO), dimethylformamide (DMF), ethyl acetate, n-methyl pyrrolidone (NMP), dimethylacetamide (DMA), and propylene carbonate.

As used herein, the term “aprotic polar solvent system” refers to a solution wherein the solvent is a single aprotic polar solvent (for example, neat DMSO), or a mixture of two or more aprotic polar solvents (for example, a mixture of DMSO and NMP).

As used herein, “residual moisture” may refer to the residual moisture in the drug powder following preparation by the manufacturer/supplier. Typical powders often have residual moisture contents ranging from up to 10% (w/w). When these powders are dissolved in an aprotic polar solvent system, the residual moisture in the powder is incorporated into the formulation. Additionally, the aprotic polar solvents may also contain a certain level of residual moisture. For example, a freshly opened bottle of USP-grade DMSO typically contains up to 0.1% (w/w) moisture. The residual moisture is different from “added moisture,” where water is intentionally added to the formulation, for example to serve as a co-solvent, or to depress the freezing point of the aprotic polar solvent system. Moisture may also be introduced into the formulation during addition of an ionization stabilizing excipient (for example, through addition of a mineral acid from an aqueous stock solution (e.g., 1 N HCl or H₂SO₄)). The total moisture content (% w/w, unless otherwise stated) in a formulation immediately following preparation is due to the contributions from both the residual moisture and the added moisture.

The term “about” or “approximately” or “substantially unchanged” are defined as being close to as understood by one of ordinary skill in the art, and in one non-limiting embodiment the terms are defined to be within 10%, preferably within 5%, more preferably within 1%, and most preferably within 0.5%. Further, “substantially non-aqueous” refers to less than 5%, 4%, 3%, 2%, 1%, or less by weight or volume of water.

“Pharmaceutically acceptable” ingredient, excipient or component is one that is suitable for use with humans and/or animals without undue adverse side effects (such as toxicity, irritation and allergic response) commensurate with a reasonable benefit/risk ratio.

“Pharmaceutically acceptable carrier” means a pharmaceutically acceptable solvent, suspending agent, or vehicle for delivering a drug compound of the present invention to a mammal such as a human.

As used herein an “ionization stabilizing excipient” is an excipient that establishes and/or maintains a particular ionization state for a therapeutic agent. In certain aspects the ionization stabilizing excipient can be, or includes, a molecule that donates at least one proton under appropriate conditions or is a proton source. According to the Bronsted-Lowry definition, an acid is a molecule that can donate a proton to another molecule, which by accepting the donated proton may thus be classified as a base. As used in this application, and as will be understood by the skilled technician, the term “proton” refers to the hydrogen ion, hydrogen cation, or H⁺. The hydrogen ion has no electrons and is composed of a nucleus that typically consists solely of a proton (for the most common hydrogen isotope, protium). Specifically, a molecule that can donate at least one proton to the therapeutic agent is considered an acid or proton source, regardless of whether it is completely ionized, mostly ionized, partially ionized, mostly unionized, or completely unionized in the aprotic polar solvent.

As used herein a “mineral acid” is an acid that is derived from one or more inorganic compounds. Accordingly, mineral acids may also be referred to as “inorganic acids.” Mineral acids may be monoprotic or polyprotic (e.g., diprotic, triprotic, etc.). Examples of mineral acids include hydrochloric acid (HCl), sulfuric acid (H₂SO₄) and phosphoric acid (H₂PO₄).

As used herein an “organic acid” is an organic compound with acidic properties (i.e. can function as a proton source). Carboxylic acids are one example of organic acids. Other known examples of organic acids include, but are not limited to, alcohols, thiols, enols, phenols, and sulfonic acids. Organic acids may be monoprotic or polyprotic (e.g. diprotic, triprotic, etc.)

“Charge profile,” “charge state,” “ionization,” “ionization state,” and “ionization profile” may be used interchangeably and refer to the ionization state due to protonation and/or deprotonation of the peptide's ionogenic groups.

As used herein, a “co-formulation” is a formulation that contains two or more therapeutic agents dissolved in an aprotic polar solvent system. The therapeutic agents may belong to the same class, or the therapeutic agents may belong to different classes.

An “amphoteric species” is a molecule or ion that can react as an acid as well as a base. These species can either donate or accept a proton. Examples include amino acids, which possess both amine and carboxylic acid functional groups. Amphoteric species further include amphiprotic molecules, which contain at least one hydrogen atom, and have the ability to donate or accept a proton.

The use of the word “a” or “an” when used in conjunction with the term “comprising” in the claims and/or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

The compositions and methods of making and using the same of the present invention can “comprise,” “consist essentially of” or “consist of” particular ingredients, components, blends, method steps, etc., disclosed throughout the specification.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa.

### DETAILED DESCRIPTION

It is well known that obesity, both in adults and children, is increasing in the United States and is becoming a substantial concern to medical professionals. In some extreme cases various types of surgery are used to reduce and control weight. The number of such surgeries has increased significantly over the past few years, to the point where approximately 200,000 surgeries are performed each year, with the number expected to continue to increase.

Gastric bypass surgery, however, is not without its complications, risks and negative consequences. One such complication is hyperinsulinemic hypoglycemia, which generally refers to after meal spikes in insulin with resulting extreme drops in blood sugar, where the patient experiences significant negative effects, including extreme sleepiness and fatigue, anxiety, in some cases a confusional state and passing out, or even seizures in extreme cases.

### I. Treatment of Post-Bariatric Hypoglycemia (PBH)

In another aspect, the present invention provides methods of treating diseases, conditions, or disorders by administering to a subject glucagon, a glucagon analog or salt thereof for treating such disease, condition, or disorder. In certain aspects the glucagon or glucagon analog can be in a stable formulation and in an amount effective to treat, alleviate, or prevent the disease, condition, or disorder. In particular embodiments the disorder is post bariatric hypoglycemia (PBH).

In some embodiments, a therapeutic method of the present invention comprises treating, ameliorating, or preventing hypoglycemia by administering to a subject having or at risk of developing PBH an effective amount of a glucagon or a glucagon analog or salt thereof. The subject can be identified as having or at risk of developing PBH by glucose monitoring.

Administered dosages of glucagon, glucagon analog, or salts thereof for treating PBH are in accordance with dosages and scheduling regimens described herein. General guidance for appropriate dosages of all pharmacological agents used in the present methods is provided in Goodman and Gilman's The Pharmacological Basis of Therapeutics, 11th Edition, 2006, supra, and in a Physicians' Desk Reference (PDR), for example, in the 65th (2011) or 66th (2012) Eds., PDR Network, LLC, each of which is hereby incorporated herein by reference. The appropriate dosage for treating PBH will vary according to several factors, including the formulation of the composition, patient response, the severity of the condition, the subject's weight, and the judgment of the prescribing physician. Effective doses of the described formulations deliver a medically effective amount of glucagon, glucagon analog, or a salt thereof. The dosage can be increased or decreased over time, as required by an individual patient or determined by medical personnel.

Nonetheless, an alert value can be defined that draws the attention of both patients and caregivers to the potential harm associated with hypoglycemia. In certain aspects an alert value can be a falling blood glucose levels below 90, 80, 70, 60, or 50 mg/dL. In a further aspect the alert value can be a falling blood glucose levels below 100, 90, 80, 70, 60, or 50 mg/dL that decreases by 1, 2, 3, 4, 5, 6, 7, 8, 9, 10 mg/dL or more over a period of time (e.g., within 90, 80, 70, 60, 50, 40, 30, 25, 20, 15, 10, 9, 8, 7, 6, 5, 4, 3, 2, or 1 minute(s)) after a meal. Patients at risk for hypoglycemia (i.e., those that have had bariatric surgery and previous episodes of hypoglycemia) should be alert to the possibility of developing hypoglycemia at a self-monitored plasma glucose—or continuous glucose monitored subcutaneous glucose—concentration of≤70 mg/dL. (≤3.9 mmol/L).

The condition of severe hypoglycemia is an event requiring assistance of another person to actively administer carbohydrates, glucagon, or take other corrective actions. Plasma glucose concentrations may not be available during an event, but neurological recovery following the return of plasma glucose to normal is considered sufficient evidence that the event was induced by a low plasma glucose concentration. Typically, these events begin occurring at plasma glucose concentrations of ≤50 mg/dL (2.8 mmol/L). Documented symptomatic hypoglycemia is an event during which typical symptoms of hypoglycemia are accompanied by a measured plasma glucose concentration≤70 mg/dL (≤3.9 mmol/L). Asymptomatic hypoglycemia is an event not accompanied by typical symptoms of hypoglycemia but with a measured plasma glucose concentration≤70 mg/dL (≤3.9 mmol/L). Probable symptomatic hypoglycemia is an event during which symptoms typical of hypoglycemia are not accompanied by a plasma glucose determination but that was presumably caused by a plasma glucose concentration≤70 mg/dL (≤3.9 mmol/L). Pseudo-hypoglycemia is an event during which the person with diabetes reports any of the typical symptoms of hypoglycemia with a measured plasma glucose concentration>70 mg/dL (>3.9 mmol/L) but approaching that level.

In light of the current specification a determination of an effective amount or dose is well within the capability of those skilled in the art. Generally, the formulations to deliver these doses may contain glucagon, glucagon analog, or salt thereof present at a concentration from about 0.1 mg/mL up to the solubility limit of the therapeutic agent in the formulation. This concentration is preferably from about 1 mg/mL to about 100 mg/mL. In certain aspects the concentration is about 1 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, or about 100 mg/mL.

The formulations of the present invention may be for subcutaneous, intradermnal, or intramuscular administration (e.g., by injection or by infusion). In some embodiments, the formulation is administered subcutaneously. The formulations can also be delivered transdermally, such as by topically applying the composition to skin (e.g., spreading the composition on skin or loading the composition onto a dermal patch and attaching the dermal patch to the skin).

Glucagon or glucagon analog formulations can be administered by infusion or by injection using any suitable device. For example, a formulation may be placed into a syringe (e.g., a pre-filled syringe), a pen injection device, an auto-injector device, or a pump device. In some embodiments, the injection device is a multi-dose injector pump device or a multi-dose pen device. The formulation is presented in the device in such a fashion that the formulation is readily able to flow out of the needle upon actuation of an injection device, such as an auto-injector, in order to deliver the therapeutic agent. Suitable pen/auto injector devices include, but are not limited to, those pen/auto injection devices manufactured by Becton-Dickenson, Swedish Healthcare Limited (SHL Group), YpsoMed Ag, and the like. Suitable pump devices include, but are not limited to, those pump devices manufactured by Tandem Diabetes Care, Inc., Delsys Pharmaceuticals, Insulet Corp. and the like.

In some embodiments, the glucagon or glucagon analog formulations are provided as ready for administration in a vial, a cartridge, or a pre-filled syringe.

Certain aspects of the methods described herein can be implemented using a pump-based system. Pump-based systems used to administer the glucagon compositions can include closed-loop, open-loop, or no-loop systems. In certain aspects glucagon or glucagon analog formulations can be used with such systems that are designed to be carried or stored in a pump container without having to be reconstituted (i.e., they are readily available to be administered to the patient/subject from the pump container). Further, the formulations can be stable at non-refrigerated temperatures (20-35° C.) for extended periods (>2 months) (i.e., the formulations can be safely stored in the pump container without risking substantial loss in activity of the glucagon in the formulation or risking the formation of insoluble aggregates than will inhibit delivery and clog the infusion apparatus).

The pump-based system can include: (1) a glucose sensor that is or can be inserted in a patient and that is capable of measuring blood glucose levels (e.g., either directly via contact with the patient's blood or indirectly via contact with the patient's interstitial fluid); (2) a transmitter that sends the glucose information from the sensor to a monitor (e.g., via radio frequency transmission); (3) a pump that is designed to store and deliver the glucose formulation to the patient; and/or (4) a monitor (e.g., one that can be built into the pump device or a stand-alone monitor) that displays or records glucose levels. For a closed-loop system, the glucose monitor can be capable of modifying the delivery of the glucagon formulation to the patient via the pump based upon an algorithm. Such a closed-loop system requires little to no input from the patient and instead actively monitors blood glucose levels and administers the needed amount of the glucagon formulation to the patient to maintain an appropriate glucose level and prevent the occurrence of hypoglycemia. For an open-loop system, the patient would actively participate by reading their glucose monitor and adjusting the delivery rate/dose based on information provided by the monitor. For a no-loop system, the pump would deliver the glucagon formulation at a fixed (or basal) dose. The no-loop system can be used without a glucose monitor and without a glucose sensor if so desired.

Certain aspects include a glucagon delivery apparatus comprising a reservoir containing a composition comprising glucagon, a glucagon analog, or a salt form thereof, a sensor configured to measure a patient's blood glucose level, and an electronic pump configured to intradermally, subcutaneously or intramuscularly deliver at least a portion of the composition to a patient based on the patient's measured blood glucose level. The sensor can be positioned on the patient such that it contacts the patient's blood or contacts the patient's interstitial fluid or both. The sensor can be configured to transmit data (for example, wirelessly, via radio frequency or bluetooth low energy (BLE), or via a wired connection) to a processor configured to control operation of the electronic pump. The processor can be configured to control operation of the pump based, at least in part, on the data obtained by the sensor. In one instance, the processor can be configured to control operation of the pump to intradermally, subcutaneously or intramuscularly inject at least a portion of the composition if the data obtained by the sensor indicates a glucose level below a defined threshold or indication that a defined threshold will be breached in a particular period of time (e.g., an indication of impending hypoglycemia or an indication that the blood glucose levels will fall to below 70, 60, or 50 mg/dL within a certain period of time (e.g., within 30, 25, 20, 15, 10, 9, 8, 7, 6, 5, 4, 3, 2, or 1 minute(s)). Such an indication can be determined by identifying a downward trend of blood glucose levels (e.g., by the blood glucose monitoring device) as well as the speed or trajectory of this downward trend. The glucagon delivery apparatus can also include a monitor configured to communicate information indicative of the patient's glucose level. The monitor can include a speaker or a display device, or both. The monitor can be configured to communicate an alert when a glucose level of the patient is estimated to be at a defined threshold. Still further, the apparatus can be configured to allow manual adjustment of at least one of a delivery rate and a dose of the composition intradermally, subcutaneously or intramuscularly delivered by the pump.

In some embodiments, the stable formulation is used for formulating a medicament for the treatment of hypoglycemia. In some embodiments, the stable formulation comprises glucagon, glucagon analog, or a salt thereof (e.g., glucagon acetate).

### II. Glucagon and Glucagon Analog Formulations

Therapeutic agents, such as glucagon and glucagon analogs, in the context of the present invention encompass peptide or protein compounds and pharmaceutically acceptable salts thereof. In certain aspects, when the therapeutic agent is present in the deoxygenated aprotic polar solvent, the stability of the therapeutic agent may be further enhanced when compared with the same therapeutic agent present in an untreated aprotic polar solvent. The increased stability can be attributed due, at least in part, to a reduction in the oxidative degradation of the therapeutic agent or the oxidative degradation of the aprotic polar solvent, or both. One of skill is aware of which therapeutic agent is suitable for treating certain diseases or conditions and would be capable of administering effective amounts of a therapeutic agent in a formulation as described herein for the treatment of a disease or condition.

Non-limiting examples of peptides and proteins and salts thereof) that can be used in the context of the present invention include, but are not limited to glucagon or analogs thereof.

The therapeutic agent of the invention can be administered intracutaneously in the prevention, diagnosis, alleviation, treatment, or cure of disease. Examples of proteins and proteinaceous compounds which may be formulated and employed in the delivery system according to the present invention include those proteins which have biological activity, or which may be used to treat a disease or other pathological conditions.

Any suitable dosage of peptide or peptides can be formulated, Generally, the peptide (or, in embodiments comprising two or more peptides, each of the peptides) is present in the formulation in an amount ranging from about 0.1 mg/mL to about 100 mg/mL. In some embodiments, the peptide is present in the formulation in an amount ranging from about 5 mg/mL, to about 60 mg/mL. In other embodiments, the peptide is present in the formulation in an amount ranging from about 10 mg/mL to about 50 mg/mL. In still other embodiments, the peptide is present in the formulation in an amount ranging from about 1 mg/mL to about 15 mg/mL. In yet other embodiments, the peptide is present in the formulation in an amount ranging from about 0.5 mg/mL to about 5 mg/mL. In yet other embodiments, the peptide is present in the formulation in an amount ranging from about 1 mg/mL, to about 50 mg/mL.

In some embodiments, the formulations can further comprise an antioxidant. In other embodiments, the formulations can further comprise a chelator. In still other embodiments, the formulations can further comprise a preservative.

Formulations used in the described therapies and methods include a glucagon or a glucagon analog or salt thereof present in an aprotic polar solvent system. In particular aspects aprotic polar solvent system includes at least one ionization stabilizing excipient. The glucagon or a glucagon analog or salt thereof can be dissolved (e.g., fully or partially solubilized) or suspended (fully or partially) in the aprotic polar solvent system. Further, the formulation can be structured as a single phase solution, a paste or slurry, a gel, an emulsion, or a suspension.

In some embodiments, the glucagon, glucagon analog or salt thereof is present in an aprotic polar solvent that is “neat,” i.e., it does not contain a co-solvent. In other embodiments the glucagon, glucagon analog or salt thereof is present in a solvent system that is a mixture of two or more aprotic polar solvents (i.e., an aprotic polar solvent system). An example would be a 75/25 (% v/v) mixture of DMSO and NMP. In some embodiments, a co-solvent can be used, where in one or more aprotic polar solvents are mixed with a co-solvent. Non-limiting examples of co-solvents include water, ethanol, propylene glycol (PG), glycerol, and mixtures thereof. In certain aspects water can be specifically excluded or limited as a co-solvent, i.e., the co-solvent can be a non-aqueous co-solvent. The co-solvent may be present in the formulation in an amount ranging from about 0.5% (w/v) to about 50% (w/v), e.g., about 1%, about 5%, about 10%, about 15%, about 20%, about 25%, about 30%, about 35%, or about 40% (w/v). In some embodiments, the co-solvent is present in the formulation in an amount ranging from about 10% (w/v) to about 50% (w/v), from about 10% (w/v) to about 40% (w/v), from about 10% (w/v) to about 30% (w/v), from about 10% (w/v) to about 25% (w/v), from about 15% (w/v) to about 50% (w/v), from about 15% (w/v) to about 40% (w/v), from about 15% (w/v) to about 30% (w/v), or from about 15% (w/v) to about 25% (w/v).

Still further, a glucagon or glucagon analog formulation can include one or more excipients. In some embodiments, the excipient is selected from sugars, starches, sugar alcohols, antioxidants, chelators, and preservatives. Examples of suitable sugars excipients include, but are not limited to trehalose, glucose, sucrose, etc. Examples of suitable starches for stabilizing excipients include, but are not limited to, hydroxyethyl starch (HES). Examples of suitable sugar alcohols (also referred to as polyols) for stabilizing excipients include, but are not limited to, mannitol and sorbitol. Examples of suitable antioxidants include, but are not limited to, ascorbic acid, cysteine, methionine, monothioglycerol, sodium thiosulphate, sulfites, BHT, BHA, ascorbyl palmitate, propyl gallate, N-acetyl-L-cysteine (NAC), and Vitamin E. Examples of suitable chelators include, but are not limited to, EDTA, EDTA disodium salt (edetate disodium), tartaric acid and salts thereof, glycerin, and citric acid and salts thereof. Examples of suitable inorganic salts include sodium chloride, potassium chloride, calcium chloride, magnesium chloride, calcium sulfate, and magnesium sulfate. Examples of suitable preservatives include, but are not limited to, benzyl alcohols, methyl parabens, propyl parabens, and mixtures thereof. Additional formulation components include local anesthetics, such lidocaine or procaine. In some embodiments, an additional stabilizing excipient is present in the formulation in an amount ranging from about 0.05% (w/v) to about 60% (w/v), from about 1% (w/v) to about 50% (w/v), from about 1% (w/v) to about 40% (w/v), from about 1% (w/v) to about 30% (w/v), from about 1% (w/v) to about 20% (w/v), from about 5% (w/v) to about 60% (w/v), from about 5% (w/v) to about 50% (w/v), from about 5% (w/v) to about 40% (w/v), from about 5% (w/v) to about 30% (w/v), from about 5% (w/v) to about 20% (w/v), from about 10% (w/v) to about 60% (w/v), from about 10% (w/v) to about 50% (w/v), from about 10% (w/v) to about 40% (w/v), from about 10% (w/v) to about 30% (w/v), or from about 10% (w/v) to about 20% (w/v). In some embodiments, the additional stabilizing excipient is present in the formulation in an amount that is about, at most, or at least 0.1, 0.5, 1, 2, 3, 4, 5, 6, 8, 9, 10, 15, 20, 25, 30, 35, 40, 45, 50, 55, or 60% (w/v).

### III. Kits/Containers

Kits are also contemplated as being used in certain aspects of the present invention. For instance, a formulation of the present invention can be included within a kit. A kit can include a container. In one aspect, for instance, the formulation can be comprised within a container that is ready to administer to a subject without having to reconstitute or dilute the formulation. That is, the formulation to be administered can be stored in the container and be readily used as needed. The container can be a device. The device can be a syringe (e.g. pre-filled syringe), a pen injection device, an auto-injector device, a device that can pump or administer the formulation (e.g., automatic or non-automatic external pumps, implantable pumps, etc.) or a perfusion bag. Suitable pen/auto-injector devices include, but are not limited to, those pen/auto-injection devices manufactured by Becton-Dickenson, Swedish Healthcare Limited (SHL Group), YpsoMed Ag, and the like. Suitable pump devices include, but are not limited to, those pump devices manufactured by Tandem Diabetes Care, Inc., Delsys Pharmaceuticals, Insulet Corp., and the like.

### EXAMPLES

The following examples are provided to better illustrate the claimed invention and are not intended to be interpreted as limiting the scope of the invention. To the extent that specific materials or steps are mentioned, it is merely for purposes of illustration and is not intended to limit the invention. One skilled in the art may develop equivalent means or reactants without the exercise of inventive capacity and without departing from the scope of the invention.

### Example 1

### Randomized, Placebo-Controlled, Double Blind, 2-Way Crossover Study Followed by an Open-Label Crossover Extension With Standard of Care, to Evaluate The Incidence and Duration of Post-Prandial Hypoglycemia in Post-Bariatric Surgery Patients

The study described has a primary objective of evaluating the incidence and duration of hypoglycemia after having meals (PG less than 70 mg/dL). And as a secondary objective assessing the (i) prevention of post prandial hypoglycemia episodes (defined as glucose levels below 70 mg/dl); (ii) prevention of severe hypoglycemia episodes (defined as glucose levels below 54 mg/dl); and prevention of rebound hyperglycemia after administration of study drug (defined as glucose levels above 180 mg/dl). Also the time in goal range will be evaluated (defined as glucose levels within 70-180 mg/dl), reported in minutes; as wells the neurogenic symptoms of hypoglycemia (if present) as documented using the Edinburgh Hypoglycemia Symptoms Score.

Other objective include: Assessing (i) the ability of a patient to self-administer glucagon using vial and syringe after CGM alert, in the open-label arm; (ii) patient satisfaction with vial and syringe format, at the end of the open-label arm; (iii) patient quality-of-life comparison measured by [EQ-5D/SF-36/etc.] between glucagon and standard-of-care in the open-label period; and (iv) fear of hypoglycemia at baseline and at the end of open-label study. Oral carbohydrate utilization in the out-patient setting.

Subjects will be adult male or female patients with post-bariatric surgery hypoglycemia syndrome defined as minimum of at least one hypoglycemic episode per week requiring intervention. Approximately 35 subjects are anticipated to be screened for this study to achieve the goal of 24 subjects completing the study with evaluable results for all treatment periods. To allow for possible drop-outs, approximately 28 subjects may be randomized.

This is an inpatient, randomized, placebo-controlled, blinded, two-way crossover study, followed by an open-label two-way crossover study in an outpatient setting, to evaluate efficacy and safety of administering a glucagon formulation in subjects with PBHS.

Subject should stop their current off-label medication for PBH for 24 hours before the treatment visit. If the subject is on LAR depot octreotide, they should be converted to an immediate acting octreotide which is stopped 24 hours before the treatment visit.

The study will involve two daytime clinical research center (CRC, or comparable setting) mixed meal tolerance test (MMTT) sessions 7-28 days apart, with random assignment to receive glucagon 300 mcg during one session and placebo during the other.

In-patient blinded visits will be followed by a 6-week open-label outpatient treatment. Subjects in this outpatient two-arm crossover study will be randomized to receive both 3 weeks of self-administered glucagon 300 mcg as needed using a vial and syringe, and 3 weeks of standard of care.

The estimated duration of study participation for individual subjects is approximately 4 weeks in clinical research center and 6 weeks in open label. The estimated duration of the entire study is 6 months.

Inclusion Criteria. Males or females diagnosed with ongoing post-bariatric surgery hypoglycemia with prior episodes of hypoglycemia, unresponsive to dietary intervention (low glycemic index, controlled carbohydrate portions) and oral acarbose. History of bariatric surgery 6 months prior to enrollment. Minimum one episode of hypoglycemia per week requiring intake of oral carbohydrates. Age 18-65 years of age, inclusive, at screening. Willingness to follow all study procedures, including attending all clinic visits.

Exclusion Criteria. Documented hypoglycemia occurring in the fasting state (>12 hours fast); Chronic kidney disease stage 4 or 5; Hepatic disease, including serum ALT or AST greater than or equal to 3 times the upper limit of normal; hepatic synthetic insufficiency as defined as serum albumin<3.0 g/dL; or serum bilirubin>2.0; Congestive heart failure, NYHA class, III or IV; History of myocardial infarction, unstable angina or revascularization within the past 6 months; History of a cerebrovascular accident in past 6 months or with major neurological deficits; Seizure disorder (other than with suspect or documented hypoglycemia); Active treatment with any diabetes medications except for acarbose; Active malignancy, except basal cell or squamous cell skin cancers; Personal or family history of pheochromocytoma or disorder with increased risk of pheochromocytoma (MEN 2, neurofibromatosis, or Von Hippel-Lindau disease); Known insulinoma; Major surgical operation within 30 days prior to screening; Hematocrit below 33%; Bleeding disorder, treatment with warfarin, or platelet count<50,000; Blood donation (1 pint of whole blood) within the past 2 months; Active alcohol abuse or substance abuse; Current administration of oral or parenteral corticosteroids; Pregnancy and/or Lactation: For women of childbearing potential: there is a requirement for a negative urine pregnancy test and for agreement to use contraception and to refrain from breast-feeding during the study and for at least 1 month after participating in the study. Acceptable contraception includes birth control pill/patch/vaginal ring, Depo-Provera, Norplant, an IUD, the double barrier method (the woman uses a diaphragm and spermicide and the man uses a condom), or abstinence; Use of an investigational drug within 30 days prior to screening.

There will be no involvement of special vulnerable populations such as pregnant women, prisoners, institutionalized or incarcerated individuals, or others who may be considered vulnerable populations.

Summary of Protocol. Visit 1—Screening. Adult male or female patients with PBHS will be recruited from multiple clinical research centers. Patients will undergo a history and physical examination, with emphasis on inclusion and exclusion criteria. Blood and urine samples will be obtained for screening laboratory testing including hemoglobin A1c, CBC, comprehensive chemistry, urinalysis, and pregnancy test (if applicable). Fear of hypoglycemia score will be recorded. Consent forms will be reviewed in detail with potential participants. Subjects will be screened within 30 days of visit 2.

Washout period. Subject should stop their current off label medication for PBHS for 24 hours before the treatment visit. If subject is on LAR depot octreotide, they should be converted to an immediate acting octreotide which is stopped 24 hours before the treatment visit.

Visit 2—CGM Sensor Placement: Two continuous glucose monitor sensors (Dexcom® G4) will be placed on the anterior abdominal wall (to ensure sensor availability and calibration for visit day. Participants will be provided a glucometer and instructed in both sensor insertion and calibration techniques. If patient has prior experience with sensor insertion and calibration, then this visit may occur concurrent with visit 1.

Visit 3—Mixed Meal Testing—Treatment 1 ((</=3 days following Visit 2). Subjects will arrive in the morning after an overnight fast. An intravenous line will be inserted in a vein in ant-cubital fossa for blood sampling. Placement of the Dexcom® sensor will be verified, and calibration verified using at least 2 venous blood glucose samples obtained 15 minutes apart. Two blood samples will be obtained for measurement of plasma glucose (via YSI analytical device) immediately and subsequent hormonal assays. Subject will be randomized after blood sample is collected. The subject will then be asked to drink a liquid mixed meal containing at least 60 gm of carbohydrates, e.g. 2 bottles of Ensure compact gm over 10 minutes. Blood samples will be collected for immediate (in room) glucose measurements (YSI) every 10 minutes till blood glucose reaches 110 mg/dl. Once venous glucose levels fall below 110 mg/dl, glucose will be measured at 5-minute intervals (YSI). This blood sampling series will conclude once sensor glucose levels fall to 90 mg/dl and the sensor displays “down arrows” indicating glucose levels are continuing to decrease. When the plasma glucose fall to 90 mg/dl and the sensor displays “down arrows” indicating glucose levels are continuing to decrease, the subject will be administered the blinded study drugs from a vial & syringe, via the subcutaneous route in the abdomen by a healthcare provider. Time will be reset to 0 min at the time of drug delivery. After study drug administration, plasma glucose will be measured (YSI) at 5, 10, 15, 20, 30, 45, 60, 90, and 120 minutes. Hormone profiles should be monitored at 10, 20, 30, 45, 60, 75, 90, and 120 minutes.

At any time post-dosing, if a subject exhibits signs of neuroglycopenia or glucose level falls at or below 54 mg/dl for greater than 5 minutes, a 25 mL IV bolus dose of 50% dextrose will be given. Signs and symptoms should be monitored and if the subject's condition fails to improve within 15 minutes, additional dextrose or other intervention may be given at the discretion of the investigator. At time of alarm trigger and before each administration of study drug, the Edinburgh Hypoglycemia Symptoms Score will be assessed. This will be repeated at 15, 30 and 60 minutes following study drug administration. Baseline glucose levels should be verified prior to discharge, CGM sensors will be removed. Sensors will be downloaded for subsequent analysis of appropriateness of dose timing. Blood samples collected during this visit will be processed and stored per the analytical laboratory guidelines, until analysis for hormone levels to verify typical patterns in response to meal ingestion and to assess endogenous responses to the study drug

Visit 4—CGM Sensor Placement [After a wash-out period of 7 to 28 days]: Two continuous glucose monitor sensors (Dexcom® G4) will be placed on the anterior abdominal wall (to ensure sensor availability and calibration for visit day). Participants will be provided a glucometer and instructed in both sensor insertion and calibration techniques. If patient is already trained on CGM sensor insertion and calibration, this visit is not required.

Visit 5—Mixed Meal Testing—Treatment 2 ((</=3 days following Visit 4). After a wash-out period of 7 to 28 days, subjects will return to the clinic and the study procedures will be repeated with each subject crossed over to the other treatment arm. After study-related procedures are performed on each of the treatment days, subjects will be trained on open-label extension study procedures before discharge.

Open-Label Two-arm Crossover Study Extension. Study staff will discuss open-label procedures with each patient. Patients will be trained to self-administer 300 mcg of glucagon using a vial & syringe via the subcutaneous route in the abdomen to treat hypoglycemia. This typically occurs 60-90 minutes following a post-prandial spike in glucose levels. Glucagon administration should occur when there is an alert from CGM at 90 mg/dl and the sensor displays “down arrows” indicating glucose levels are continuing to decrease. The patient will be discharged home into the open-label study with 1 new CGM & 6 sensors. Patient should replace every week a calibrate daily using blood glucose meter, per manufacture instructions. Subject should enter all information in e-dairy, and the CRC will follow up every week to ensure sensor replacement & calibration has occurred. Subject will be randomized to either RTU-Glucagon or standard of care (SOC) treatment for 3 weeks, followed by 3 weeks with the other treatment. Two glucagon emergency kits (GEK) will be provided to each subject should severe hypoglycemia persist despite treatment with experimental drug or oral glucose (in SOC arm). If the glucose level falls at or below [54 mg/dl], or if the patient develops neuroglycopenia or discomfort with signs and symptoms of hypoglycemia, patient should self-treat with a provided GEK emergency kit and glucose tablets. Patient will return home with one attached CGM & sensor, with appropriate hypoglycemia alarms set as determined by the clinician. The CGM sensors will be replaced and recalibrated per manufacturer label across the 6-week outpatient study. Patient should record all hypoglycemia events and subsequent treatments (glucagon and/or oral carbohydrate) in an e-diary.

Visit 6—End of Study Safety Follow-up [42 to 49 days following visit 5]: At the end of 6 weeks patients will return to the clinic, CGM, sensors will be removed and data downloaded. E-diary and all data forms will be received and inspected by study staff. Patients will undergo a brief physical examination and any AEs that occurred during the outpatient study will be reviewed. Blood and urine samples will be obtained for screening laboratory testing including hemoglobin A1c, CBC, comprehensive chemistry, urinalysis, and pregnancy test (if applicable). Fear of hypoglycemia score following use of investigation drug will be recorded.

The primary endpoint will be the treatment effect on glucose levels within the lab study, as measured by YSI 2300 and/or YSI 2900 and the treatment effect on glucose levels within the open-label study, as measured by CGM. Other secondary endpoints can include glucose levels below, within, or above target range after study drug administration in lab measure YSI, measured by CGM during open-label study and defined as area under the curve (AUC) and area over the curve (AOC) in minutes: Below range, as defined by plasma glucose 70 mg/dl. Below range, as defined by plasma glucose between 54-70 mg/dl. Below range, as defined by plasma glucose<54 mg/dl. Within range, as defined by plasma glucose between 70-180 and/dl. Above range, as defined by plasma glucose>180 mg/dl. As well as the treatment effect on symptoms of hypoglycemia, as measured by [Edinburgh Hypoglycemia Symptoms Score] during the inpatient lab study.

Exploratory endpoints can include: Usability of the vial and syringe, as measured by [XERIS questionnaire?] at end of open-label study. Quality of Life index, measured by [EQ-5D/SF-36/etc.] at baseline and at the end of open-label study. Hormone profile (Insulin and glucagon) during the inpatient portion of the study. Fear of hypoglycemia at baseline and at the end of open-label study. Carbohydrate utilization compared between study drug and SOC.

1-35. (canceled)

36. A method of preventing or treating hypoglycemia in a post-bariatric surgery subject comprising:
(a) determining whether or not the post-bariatric surgery subject is at risk of developing post-bariatric hypoglycemia (PBH); and
(b) administering a therapeutic formulation comprising a glucagon peptide, a glucagon analog, or salts thereof to the subject if the subject is determined to be at risk of developing PBH.

37. The method of claim 36, further comprising monitoring the subject's blood glucose levels.

38. The method of claim 36, wherein the hypoglycemia is a post-prandial hypoglycemia episode.

39. The method of claim 36, wherein the hypoglycemia is a severe hypoglycemia episode.

40. The method of claim 38, wherein the subject's post-prandial blood glucose levels are decreasing and are below 100, 90, 80, 70, 60, or 50 mg/dL.

41. The method of claim 40, wherein the subject's blood glucose falls below 100, 90, 80, 70, 60, or 50 mg/dL within 90, 80, 70, 60, 50, 40, 30, 25, 20, 15, 10, 9, 8, 7, 6, 5, 4, 3, 2, or 1 minutes after a meal.

42. The method of claim 36, wherein the therapeutic formulation is administered 10 to 90 minutes after a meal.

43. The method of claim 37, wherein the therapeutic formulation is administered when the subject's blood glucose decreases by 0.5 to 10 mg/dL/min 90, 80, 70, 60, 50, 40, 30, 25, 20, 15, 10, 9, 8, 7, 6, 5, 4, 3, 2, or 1 minutes after a meal.

44. The method of claim 36, wherein the therapeutic formulation comprises 50 to 300 μg of a glucagon peptide, a glucagon analog, or salts thereof.

45. The method of claim 36, wherein the therapeutic formulation comprises 150 μg of a glucagon peptide, a glucagon analog, or salts thereof.

46. The method of claim 36, wherein the therapeutic formulation is administered as a bolus.

47. The method of claim 36, wherein the therapeutic formulation is administered as an infusion over 90 seconds to 45 minutes.

48. The method of claim 36, wherein the therapeutic formulation is administered from a delivery apparatus.

49. The method of claim 48, wherein the delivery apparatus comprises: (i) a reservoir containing the therapeutic formulation; and (ii) an electronic pump configured to intradermally, subcutaneously, or intramuscularly deliver at least a portion of the therapeutic formulation to the subject.

50. The method of claim 48, wherein the apparatus is a closed-loop system, an open-loop system, or a no-loop system for delivering the therapeutic formulation to the subject.

51. The method of claim 36, wherein the therapeutic formulation is a single-phase solution comprising the glucagon peptide, glucagon analog, or salts thereof, dissolved in a non-aqueous solvent.

52. The method of claim 51, wherein the non-aqueous solvent is an aprotic polar solvent.

53. The method of claim 52, wherein the aprotic polar solvent is dimethylsulfoxide (DMSO).

54. The method of claim 52, wherein the aprotic polar solvent is a deoxygenated aprotic solvent.

55. The method of claim 52, wherein the therapeutic formulation further comprises an ionization stabilizing excipient, wherein (i) the glucagon peptide, glucagon analog, or salts thereof is dissolved in the aprotic polar solvent in an amount from about 0.1 mg/mL up to the solubility limit of the glucagon peptide, glucagon analog, or salts thereof, and (ii) the ionization stabilizing excipient is dissolved in the aprotic solvent in an amount to stabilize the ionization of the glucagon peptide, glucagon analog, or salts thereof.

56. The method of claim 55, wherein the ionization stabilizing excipient is at a concentration of 0.1 mM to less than 100 mM.

57. The method of claim 55, wherein the ionization stabilizing excipient is a mineral acid.

58. The method of claim 57, wherein the mineral acid is sulfuric acid or hydrochloric acid.

59. The method of claim 57, wherein the mineral acid is sulfuric acid.

60. The method of claim 55, wherein the ionization stabilizing excipient is sulfuric acid and the aprotic polar solvent is DMSO.

61. The method of claim 55, wherein the therapeutic formulation has a moisture content of less than 10, 5, or 3%.

62. The method of claim 55, wherein the therapeutic formulation further comprises a preservative at less than 10, 5, or 3% w/v.

63. The method of claim 62, wherein the preservative is benzyl alcohol.

64. The method of claim 55, wherein the therapeutic formulation further comprises a sugar alcohol at less than 10, 5, or 3% w/v.

65. The method of claim 64, wherein the sugar alcohol is mannitol.

66. The method of claim 55, wherein the therapeutic formulation further comprises at least one carbohydrate.

67. The method of claim 66, wherein the carbohydrate is trehalose and/or mannitol.

68. The method of claim 55, wherein the therapeutic formulation comprises at least 80 wt. % of the aprotic polar solvent, 3 to 7 wt. % of the carbohydrate, 0.001 to 0.1 wt. % of an amphoteric molecule, and 0 wt. % to less than 0.1 wt. % of the acid.

69. The method of claim 36, wherein the therapeutic formulation has a water content of 0 to less than 15 wt. %, 0 to less than 3 wt. %, 3 to 10 wt. %, or 5 to 8 wt. %.

70. The method of claim 36, wherein the glucagon peptide, glucagon analog, or salts thereof, has been previously dried from a buffer, wherein the dried glucagon peptide, glucagon analog, or salts thereof, has a first ionization profile that corresponds to an optimal stability and solubility for the glucagon, glucagon analog, or salt form thereof, wherein the dried glucagon peptide, glucagon analog, or salts thereof, is reconstituted into an aprotic polar solvent and has a second ionization profile in the aprotic polar solvent, and wherein the first and second ionization profiles are within 1 pH unit of one another.

71. The method of claim 49, wherein the therapeutic formulation has been stored in the reservoir for at least 1, 2, 3, 4, 5, 6, 7, 14, 21, 30, 45, or 60 days.
